(19) United States
(12) Patent Application Publication
(10) Pub. No.: US 2013/0144035 A1
(43) Pub. Date: Jun. 6, 2013

(54) COMPOSITIONS AND METHODS FOR LEUKOCYTE-TARGETING MULTI-VALENT IMAGING PROBES

(71) Applicants: Dongfeng Pan, Charlottesville, VA (US); Stuart S. Berr, Crozet, VA (US); Yi Zhang, Charlottesville, VA (US)

(72) Inventors: Dongfeng Pan, Charlottesville, VA (US); Stuart S. Berr, Crozet, VA (US); Yi Zhang, Charlottesville, VA (US)

(21) Appl. No.: 13/642,670

(22) PCT Filed: Apr. 21, 2011
(86) PCT No.: PCT/US2011/033430

§ 371 Date: Feb. 22, 2013

Related U.S. Application Data
(60) Provisional application No. 61/326,308, filed on Apr. 21, 2010.

(51) Int. Cl. A61K 51/08

(52) U.S. Cl. CPC A61K 51/088
USPC 530/323

(57) ABSTRACT

The present application discloses a new multivalent peptide ligand specifically targeting polymorphonuclear leukocytes (PMNs) with favorable pharmacological parameters to monitor sites of inflammation for imaging. The detailed synthesis, characterization, and pharmacological evaluation of the ligands are reported here. Two separate peptide binding ligands for formyl peptide and tuftsin receptors were chosen to link together based on the high expression levels of the two receptors on activated PMNs The heterobivalency and pegylated links were incorporated in the structural design to improve the sensitivity of the detection and to improve the bioavailability along with blood clearance profile, respectively. Two chemical constructs: cFLFLF-(PEG)ₙ-TKPPR-^{99m}Tc (n=4, 12) were evaluated in vitro with human PMNs for binding affinity and bioavailability. As a result, FLFLF-(PEG)₁₂-TKPPR^{99m}Tc was found to have more favorable pharmacological properties and was therefore used for further in vivo studies. Preliminary in vivo assessment of the agent was performed using Single Gamma Emission Computed Tomography (SPECT) imaging of a mouse model of ear inflammation. The results of these studies indicate cFLFLF-(PEG)₁₂-TKPPR-^{99m}Tc may be a desirable imaging agent for binding to PMNs to identify sites of inflammation by SPECT.

### BACKGROUND

High-quality non-invasive nuclear and optical imaging can be a useful diagnostic and prognostic tool for detecting sites of inflammation. This should aid in designing therapies to control pathological conditions. However, the ability to detect and characterize inflammation has been so far elusive (1). One of the hallmarks of inflammation is migration and activation of leukocytes. Current leukocyte imaging techniques include ex vivo white blood cells labeling with ⁶⁷Ga citrate, ¹¹¹In or ^{99m}Tc containing complexes and re-injection of the labeled cells back into patients. Although the utility of this method has been proven, the process is laborious and involves handling of blood products, thereby increasing the risk of contamination. Tracking neutrophils by intravenous administration of highly specific radionuclide or near infrared fluorophore (NIF) labeled binding probes would circumvent these limitations. As a result, numerous chemotactic peptides, such as fMLF, and its analogs have been investigated as potential neutrophil imaging probes (2, 3). However, exhibition of undesired biological side effects and/or poor pharmacokinetic parameters have limited their clinical utility.

We recently reported that a peptide sequence, cFLFLF, after conjugated with a polyethyleneglycol moiety (PEG) and a nuclear isotope or a NIF, could be used to detect neutrophil activation and migration (4-6). However, it is believed that multivalency can further improve the affinity and specificity of synthetic ligands to their biological targets (7-12).

There is a long felt need in the art for high specific and more sensitive imaging agents. The present invention satisfies this need.

### SUMMARY OF THE INVENTION

The present invention provides compositions and methods useful for preparing high sensitive and specific multivalent imaging probes (13). In one aspect, the probes are useful for detecting and monitoring inflammation.

To enhance the detection sensitivity, binding affinity and specificity of the peptide ligand towards neutrophils, we designed and evaluated a heterobivalent peptide, cFLFLF-PEG-TKPPR-^{99m}Tc, that simultaneously targets the FPR and tuftsin receptors of neutrophils (13). The heterobivalent probe demonstrated favorable imaging characteristics. Heterobivalent ligands have demonstrated higher affinity than monovalent and symmetrical bivalent binders (homodimer) (9, 14).

The two binding peptide sequences were linked through a bifunctional PEG (e.g., PEG₄ or PEG₁₂) moiety. One of ordinary skill in the art will appreciate that the invention is not restricted to the use of these two specific PEGs. An additional lysine residue was incorporated in the construct as a handle for HYNIC group and its coordination to radiometal (FIG. 1). The present application discloses a detailed synthetic method, the observed pharmacokinetic parameters for these new probes and a preliminary demonstration of its application to SPECT imaging of inflammation in vivo.

In one aspect, cells are targeted with the probes of the invention. In one aspect, the cells are neutrophils.

### BRIEF DESCRIPTION OF THE DRAWINGS

FIG. 1. Reagent and conditions: i) standard solid phase Fmoc chemistry; ii) (1) piperidine, (2) Fmoc-PEG-COOH, HBTU, DIEA; iii) standard solid phase Fmoc chemistry; iv) (1) 2% hydrazine, (2) 6-Boc-HYNIC acid, HBTU, DIEA; v) TFA(95%); yl) NaTcO₄, SnCl₂, Nicotinic acid, pH 5.2

FIG. 2. Monoexponential blood clearance curve of cFLFLF-PEG₁₂-TKPPR-^{99m}Tc in control mice (n=3): the normalized blood radioactivity was plotted against various time points after tail vein injection of the tracer.

FIG. 3. Tissue and organ accumulation of cFLFLF-PEG₁₂-TKPPR-^{99m}Tc at 18 hr post injection of normal mice (n=3) expressed as the percent injected dose per gram of tissue.

FIG. 4. (A) Representative MPO-BLN image of mouse subjected to topical application of PMA on left ear for 24 hours. (B) Representative transaxial images of MicroCT (left), MicroSPECT (right) and fused micro-CT and micro-SPECT (middle) of mice subjected to PMA application on left ears. Both SPECT and CT images were obtained 3 hr after tail vein injection of cFLFLF-PEG₁₂-TKPPR-^{99m}Tc. SPECT scans revealed PMA infected ear had visually more tracer uptake compared to control ear, which was quantified by ROI analysis.

FIG. 5 illustrates examples of various multi-valent imaging reagents in accordance with embodiments of the invention.

### DETAILED DESCRIPTION

### Definitions

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. For example, in one aspect, the term “about” is used herein to modify a numerical value above and below the stated value by a variance of 20%.

### EXAMPLES

### Materials and Methods.

All chemicals obtained commercially were of analytical grade and used without further purification. Na^{99m}TcO₄ was obtained from Cardinal Health INC (Charlottesville, Va.). 6-Boc-hydrazinonicotinic acid (6-Boc-HYNIC acid) was obtained from SoluLink. (San Diego, Calif.). Fmoc-Arg-PS resin was purchased from Applied Biosystems (Foster City, Calif.). Fmoc-amino acids were purchase from Anaspec Inc. (Fermont, Calif.). N-Fmoc-amino-dPEG^{TM}₄ acid and N-Fmoc-amino-dPEG^{TM}₁₂ acid were purchased from Quanta Biodesign, Ltd. (Powell, Ohio). N-hydroxysulfosuccinimide (Sulfo-NHS) and 1-ethyl-3-[3-(dimethylamino)-propyl]carbodiimide (EDC) were purchased from PIERCE (Rockford, Ill.). All other chemical reagents and solvents were obtained from Sigma-Aldrich. For purification of peptides precursors and ^{99m}Tc labeled products, semipreparative reversed-phase high-performance liquid chromatography (RP-HPLC) was performed with an Apollo C18 reversed-phase column (5μ, 250×10 mm) on a Varian system with ABI Spectroflow 783 UV detector and Bioscan NaI solid scintillation Flow Count Radio-HPLC detector.

The mobile phase was changed from 60% Solvent A (0.1% TFA in water) and 40% Solvent B (0.1% TFA in 80% aqueous acetonitrile) to 100% Solvent B at 30 mM at a flow rate 3 mL/min. MALDI-TOF mass spectroscopy analysis was performed on samples of peptide products at the W. M. Keck Biomedical Mass Spectrometry Laboratory at the University of Virginia (UVa) and the data was obtained on a Bruker Daltonics system (Billerica, Mass.). Peptides were synthesized manually by standard solid-phase method, following a conventional Fmoc strategy using 2-(1H-Benzotriazole-1-yl)-1,1,3,3-tetramethyluronium hexafluorophosphate (HBTU) as the coupling agent. Lysine was used as handle to incorporate HYNIC to the peptide. High performance liquid chromatographic (HPLC) analysis of the non-radiolabeled compounds was performed on a Varian Prostar system equipped with a ProStar 335 HPLC Diode Array Detector. HPLC solvents were purchased from Fisher Scientific (Pittsburgh, Pa.) and used without further purification. Typical yields of the crude peptides were 80-85%.

Human tumor necrosis factor-α (TNF-α) was procured from Perpotech, and fMLF was purchased from Sigma. Aliquots of both samples were taken (TNF-α, 10 U/mL, and fMLF, 10 mM) and stored at −20° C. For every assay the solutions were thawed to ambient temperature and freshly diluted with hepatic arterial (HA) buffer before use. Multiscreen high-throughput screening (HTS) with glass fiber filter (FC) 96-well plates, type C, with 1.2-mm glass filters were purchased from Millipore. Filtration from 96-well plates was performed under vacuum on a Brandel filtration device. The membranes from each well were collected by punching with the Millipore Multiscreen punching instrument. The radioactivity from ^{99m}Tc-bound ligand was measured with either Minaxi (Packard), Autogamma 5000 series (Packard), or Wallac 1420 Wizard (Perkin-Elmer) γ-counters. Radioactivity was measured for 1 min per sample and was not corrected for decay.

Human neutrophils were prepared from normal heparinized (10 U/mL) venous blood by a 1-step Ficoll-Hypaque separation procedure (15, 16), yielding approximately 98% neutrophils; greater than 95% viable as determined with trypan blue containing less than 50 pg/mL of endotoxin. After separation, neutrophils were washed with Hank's balanced salt solution with heparin (10 U/mL) 3 times. After the third wash, neutrophils were resuspended in HA buffer, which was Hank's balanced salt solution supplemented with 0.1% human albumin (Bayer Healthcare). Neutrophil experiments were completed in HA buffer.

Female FVB mice, 5 months old, were purchased from the National Cancer Institute (Frederick, Md.). Mice were housed in a controlled environment (12 h light/12 h dark photoperiod, 22±1° C., 60±10% relative humidity) and were provided free access to autoclaved pellet food and tap water. All procedures were in accordance with current National Institutes of Health (NIH) guidelines and were approved by the University of Virginia Animal Care and Use Committee (ACUC).

SPECT/CT imaging was performed with a microSPECT/CT scanner designed and built at UVa under NIH funding (17). The scanner uses an open-barrel type gantry consisting of two 38″ steel wheels connected by aluminum profile pieces. CT and SPECT scanning are performed sequentially and the animal is translated axially from one subsystem to another between two fixed locations along a 1″ diameter carbon fiber half cylinder that is located at the gantry axis of rotation. Reproducibility of animal table location permits consistent and simple fusion of CT and SPECT images using stored offset parameters. The scanner can be operated with two different types of x-ray detectors: CCD-based for high spatial resolution (modulation transfer function in the reconstructed CT image of 0.1 at a spatial frequency of 14.7 mm⁻¹ when used with an 8 micron x-ray source) or CMOS-based for fast readout (2 frames per second). Microfocus x-ray sources with focal spot sizes ranging between 8 and 50 microns are available. SPECT scanning can be performed using up to four gamma cameras (10 cm×10 cm fields of view) simultaneously. Respiratory and cardiac gating are available for both CT and SPECT scanning. CT projection images are preprocessed for detector sensitivity uniformity correction and dark count subtraction using a custom written IDL program and then are reconstructed with a Feldkamp 3-dimensional filtered backprojection algorithm (COBRA, Exxim, Inc., Pleasanton, Calif.). SPECT data are acquired using a custom written interface using Kmax (Sparrow Corp., Port Orange, Fla.) software and reconstructed using a custom written maximum-likelihood expectation-maximization algorithm (18).

Synthesis of cFLFLF-PEGₙ-K(HYNIC)TKPPR 6 and 7

(Scheme 1). Fmoc-Arg-PS resin 1 (150 mg, 0.21 mmol/g) was loaded to a peptide synthesis vessel, suspended in DMF (2 mL) and shaken at 600 rpm for 5 min and drained. Fmoc was removed by adding 2 mL of 20% piperidine-DMF solution to the synthesis vessel. The vessel was shaken on a vibrator (Labnet Shaker 20, Woodbridge, N.J.) for 5 mM and the solution was drained. The same deprotection step was repeated once again by shaking for 20 mM. The deprotected resin was washed six times each with 2 mL of DMF. The resin was then coupled to the Fmoc-protected amino acid by successively adding 4 molar excess of the Fmoc-protected amino acid (0.84 mmol) in DMF (0.1 M), 4 equivalents of HBTU in DMF (0.1 M), and 8-fold excess of DIEA (1.68 mmol). The reaction vessel was shaken for 90 min. The reaction solution was drained off and the resin was washed with 3×2 mL of DMF. The peptide resin was subsequently capped by adding 10 fold molar excess of acetic anhydride (0.5 mL) and DIEA and shaking at 600 rpm for 30 min. After draining off the solution, the resin was washed with 6×2 mL of DMF and was then ready for the next cycle of coupling. To obtain the resin-bound peptide sequence 4, the amino acids and special residues were coupled in following order: Fmoc-Pro, Fmoc-Pro, Frnoc-Lys(t-Boc), Fmoc-Thr(t-Bu), Fmoc-Lys(ivDDE), N-Fmoc-amino-dPEG^{TM}₄ acid or N-Fmoc-amino-dPEG^{TM}₁₂ acid, Fmoc-Phe, Fmoc-(D)Leu, Fmoc-Phe, Fmoc-(D)Leu, Frnoc-Phe, and Trans-cinnamic acid. The ivDDE protecting group of 4 was removed to free the ω-NH₂ of the lysine by treatment with 2% hydrazine in DMF for 60 min. After draining, the peptide resin was repeatedly washed with DMF (2 mL) for six times. To conjugate the chelating HYNIC moiety at the ω-NH₂ of the lysine a solution of 4 molar excess of 6-Boc-HYNIC acid, 4 equivalents of HBTU, and 8 equivalents of DIEA in 1.6 mL of DMF was added into the reaction vessel. The vessel was shaken for 90 mM, the solution was drained off, and the resin was washed with DMF (2 mL) for three times. The peptide cFLFLF-PEGₙ-K(HYNIC)TKPPR 6 or 7 was cleaved from the resin support using a 95% TFA. The crude products were purified with HPLC conditions described above. The molecular weights of the purified precursors were verified by MALDI-TOF Mass Spectroscopy.

cFLFLF-PEGₙ-TKPPR-^{99m}Tc 8 or 9.

To a 1.5 mL vial was consecutively added 100 μg of cFLFLF-PEG-K(HYNIC)TKPPR 6 or 7 in 200 μL of citrate buffer (10 mM, pH 5.2), 200 μL of tricine solution (30 mg/mL in 10 mM citrate buffer, pH 5.2), 100 μL of nicotinic acid solution (10 mg/mL in 10 mM citrate buffer, pH 5.2), 37 MBq of ^{99m}TcO₄⁻ solution (370 MBq/mL in saline), and 25 μL of SnCl₂ solution (1.0 mg/mL in 0.1 N HCl). The reaction mixture was stirred by shaking and heated at 60° C. for 15 min. After being cooled to room temperature for 10 mM, the reaction mixture was purified by same HPLC conditions mentioned above. The observed retention times of cFLFLF-PEG₄-TKPPR-^{99m}Tc 8 and cFLFLF-PEG₁₂-THPPK-^{99m}Tc 9 were 16.8 and 17.8 mM, respectively.

Partition Coefficient.

Freshly HPLC purified cFLFLF-PEGₙ-KTKPPR-^{99m}Tc 8 or 9 (˜350 kBq) in 500 μL of water was mixed with 500 μLof octanol in an Eppendorf microcentrifuge tube. The tube was sonicated for 10 mM and then centrifuged at 4,000 rpm for 5 mM (Fisher Scientific Marathon Micro-A). Aliquots (100 μL) of octanol and aqueous layers were carefully separated and radioactivity of samples were counted. Log P is derived as the log of the ratio of radioactivity in octonol to radioactivity in water. The measurement was repeated in triplicate and data presented is the average of three measurements.

Neutrophil Binding.

Freshly isolated human neutrophils (4×10⁶ cells/mL) were treated with TNF-α(10 U/mL, Peprotech) twenty minutes prior to binding studies and transferred to a 96 well plate (Multiscreen® HTS FC by Millipore, Billerica, Mass. 1.2 μm glass filter type C, 50.0 μL, 2.0×10⁵ cells/well). Saturation assays were carried out using eight different concentrations of cFLFLF-PEG₁₂-TKPPR-^{99m}Tc ranging from 1.0 μM to 0.01 nm incubated at 25° C. for 90 mM. Neutrophils were incubated with the excess radioligand at 25° C. for 90 minutes to obtain total binding. Following incubation, the plates were filtered rapidly under vacuum using Brandel filtration device (Brandel Inc. Gaithersburg, Md.), washed three times with cold Tris-Mg buffer (−5° C., 10 mM, 150 μL each time/well) to remove the unbound radioligand, and dried under vacuum. The plates were then wiped with Kimwipes EX-L at room temperature and membranes from each well were collected by Millipore multiscreen punching instrument (Billerica, Mass.). The bounded radioactivity remaining on the membranes was measured in a gamma counter. Specific binding was calculated as the difference between total binding and nonspecific binding at the highest concentration. Non-radioactive peptide (cFLFLF-PEG₁₂-TKPPR-^{99m}Tc) was used in excess (100 μM) to determine non-specific binding. Saturation experiment data (Kd and Bmax values) were obtained by computer analysis using PRISM 4.0 (GraphPad).

Blood Clearance.

Blood kinetics of cFLFLF-PEG₁₂-TKPPR-^{99m}Tc was studied in 3 control (non-inflamed) mice. Approximately 50 μL of blood from the contralateral tail vein were collected in capillary tubes at 15, 30, 45, 60, 120, and 180, 360 min after tracer injection (0.37-0.74 MBq). The capillary tubes were placed in a vial that was weighed beforehand and afterward. Activity in each blood sample was measured in a radioisotope calibrated well counter (CRC-15W, Capintec, Inc, NJ), decay corrected back to the time of tracer injection, normalized for injected dose and animal body weight, and expressed as % ID/g of blood.

Serum Stability of cFLFLF-PEG₁₂-TKPPR-^{99m}Tc.

Fifty microcuries of the cFLFLF-PEG₁₂-TKPPR-^{99m}Tc was added in to 100 μL of fetal bovine serum (Invitrogen, Grand Island, N.Y.). After incubation at 37° C. for 1, 3, and 6 hrs, aliquots of the mixture was filtered through a 0.2 μM micro spin filter. The filtrates were analyzed by reverse-phase HPLC with a Bioscan Flow Count Radio-HPLC detector. All of newly formed γ-peaks besides the original cFLFLF-PEG₁₂-TKPPR-^{99m}Tc peak would be considered as degraded products.

Biodistribution.

Body distribution of radioactivity was determined in control (n=3) mice 18 h after injection of tracer. After a single blood sample had been taken from the tail vein, mice were euthanized by deep halothane anesthesia. The organs and tissues (ear, heart, lungs, muscle, bone, liver, kidney, spleen, small intestine, and stomach) were collected, rinsed with PBS, wiped with filter paper and weighed in a pre-weighed vial. The radioactivity of each sample was measured in a γ-well counter and decay-corrected back to the time of tracer injection. Biodistribution values are expressed as a percentage of the injected dose (% ID) and normalized by body and organ/tissue mass.

Mouse Ear Inflammation Model.

A female FVB mouse, 5 month old, with left ear inflammatory lesion was used to preliminarily assess the ability of the cFLFLF-PEG₁₂-TKPPR-^{99m}Tc to detect inflammation as a result of neutrophils migration and accumulation. The animal model is similar to that described by Gross et al. (19). Topical application of PMA (2.5 μg in 20 μl DMSO) onto the left earlobe induces acute dermatitis, manifested by local swelling, erythema and infiltration of neutrophils (20, 21). The right ear served as control and received only DMSO (vehicle). The SPECT/CT imaging was correlated with myeloperoxidase (MPO) activity determined by non-invasive bioluminal-bioluminescence imaging (19).

Luminal-bioluminescence Imaging of MPO.

Since MPO is the most abundant protein in azurophilic granules of neutrophils, MPO specific luminal-bioluminescence study was carried out in support of the accumulation and activation of neutrophils in PMA-treated ear. The mice were injected with 200 μl of luminol (5 mg/0.1 L DMF) intraperitoneally 22 h after PMA application. Immediately after luminal injection, a 40 min kinetic bioluminescence scan was performed on Xenogen IVIS Spectrum (Caliper life science, CA) and processed on Live Image software (Caliper life science) with open filter and exposure time of 60 s.

SPECT/CT Imaging.

Twenty-four hours after PMA challenge, 3.7 MBq (1 mCi) of cFLFLF-PEG₁₂-TKPPR-^{99m}Tc in 200 μl of saline was administered via tail vein. Three hours later, CT/SPECT imaging was performed using the microSPECT/CT scanner described above. Anesthesia (1%-2% isoflurane in oxygen) was delivered throughout the imaging procedure. CT projection data acquisition used the CMOS x-ray detector and obtained 200 evenly spaced projections spanning 240 degrees over approximately 5 minutes. For SPECT scanning sixty evenly spaced views were obtained over 180 degrees using two opposing gamma cameras simultaneously, resulting in a total of 120 projections over 360 degrees at an angular increment of 3 degrees. The acquisition time was approximately 45 minutes. The two cameras were fitted with 0.5 mm diameter tungsten pinholes. The reconstructed CT voxel size was 0.164×0.164×0.164 mm³ on a 320×320×384 image matrix. The reconstructed SPECT voxel size was 0.4×0.4×0.4 mm³ on a 60×60×60 image matrix. All SPECT images were corrected for radioactivity decay but not for gamma ray attenuation.

Image Analysis.

Co-registration of SPECT and CT images was performed using ASIPRO software (Siemens Medical Solutions USA, Inc., Knoxyille, Tenn.) and a transformation matrix previously obtained with a dual modality phantom. To characterize the accumulation of the tracer in the ears, volume-of-interest (VOI) analysis was performed to identify the number of detected counts originating in the ears. CT images were used to identify voxels lying within the ear and the corresponding SPECT voxels were included in each VOI. Inflamed VOI was drawn tightly around the ear inflammation area on the SPECT transaxial image slice, and copied to the control ear. Six slices were taken in both ears. The upper 60% signal (in counts/mL) was taken into calculation in each slice. The known gamma camera detection efficiency was used to compute the activity contained in the ears. This activity was expressed as percentage of the injected dose per mouse body mass (% ID/g).

Results and Discussion

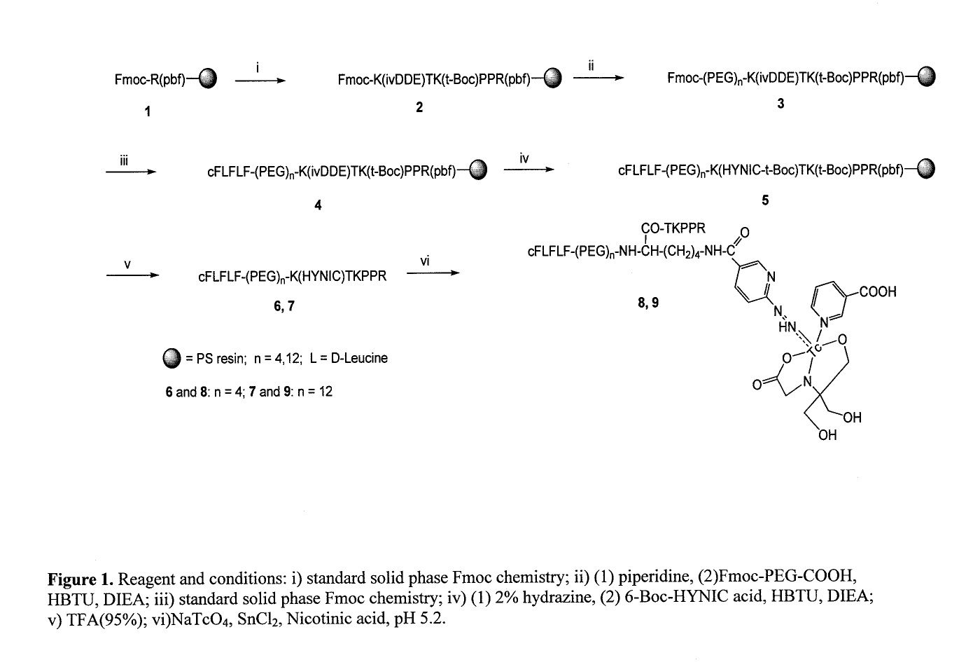

The precursors, cFLFLF-PEGₙ-K(HYNIC)TKPPR 6 and 7, were synthesized using standard Fmoc peptide chemistry on resin. Briefly, as illustrated in FIG. 1, starting from arginine residue loaded on resin at carboxyl group, the TK(t-Boc)PPR peptide was constructed first, an additional lysine residue [Lys(ivDDE)] was added to form intermediate 2 prior to addition of varied pegylated linker. Then on the other side of the PEG the second cFLFLF peptide was built to produce the desired peptide sequence 4. Finally the removal of ivDDE protecting group of lysine linker using basic hydrazine conditions released the side chain ω-NH₂ derivative 5 for conjugation with HYNIC. The resin bound HYNIC conjugated bivalent peptides 6 and 7 were cleaved with standard TFA chemistry. The two precursor peptides were purified by reverse phase HPLC with total yield in the range of 20˜25%. The purified peptide constructs were characterized by mass spectroscopy for its composition. The calculated and observed MALDI-TOF-MS [M+H]⁺ were 1905 and 1906 for cFLFLF-PEG₄-K(HYNIC)TKPPR 6 and 2258 and 2258 for cFLFLF-PEG₁₂-K(HYNIC)TKPPR 7. The two radiolabeled cFLFLF-PEGₙ-K(HYNIC)-TKPPR^{99m}Tc (8) (n=4) and (9) (n=12) were obtained by conjugating 6 and 7 with stannous chloride reduced [^{99m}Tc]pertechnedate with nicotinic acid and tricine as coligands, respectively. The radiolabeled peptides were purified from unlabeled precursors and other reagents by reverse phase semi-preparative HPLC with radiochemical yield>75% and radiochemical purity of 98%. The detailed synthetic method is described in scheme 1.

The in vitro binding studies to isolated human neutrophils exhibited Kd values 47.8 and 15.1 nM for ligand 8 and 9 respectively. The radiolabled ligand 8 was roughly 3 fold less potent than compound 9, possibly due to the longer PEG₁₂ linker allows the simultaneous binding to both the formyl peptide and the tuftsin receptors on activated neutrophils. The purified radioligands 8 and 9 were first evaluated for their Log P values to estimate approximate lipophilicity of the individual compounds. The observed Log P values for 8 and 9 are in agreement with the expected trend as PEG12 linked found to be more hydrophilic (−0.89) compared to PEG4 derivative (−0.77). Based on the better solubility in water and higher binding affinity the radioligand 9 was chosen for further in vivo studies.

The cFLFLF-PEG₁₂-TKPPR-^{99m}Tc is stable for at least 6 hours in serum. HPLC analysis of serum samples mixed with ligand displayed no appreciable fragments of ^{99m}Tc labeled peptide at each time point (1, 3, and 6 hours).

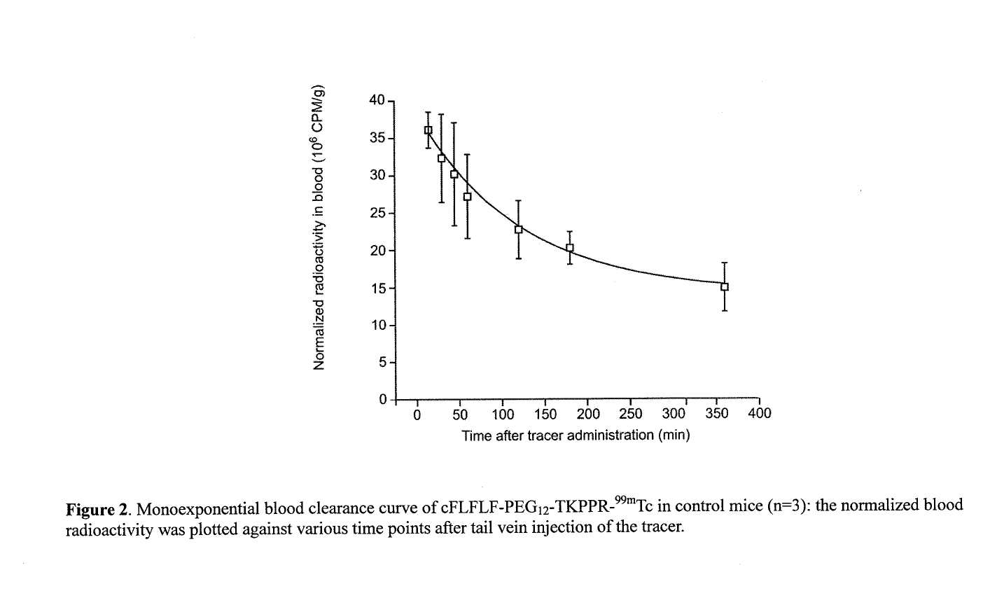

In order to further analyze the pharmacokinetic parameters of the ligand 9, blood clearance studies were conducted in vivo. As shown in FIG. 2, the data revealed a mono-exponential clearance of the radiotracer as shown in FIG. 1. The elimination half-life for blood clearance (T_{1/2}) was calculated to be about 84 min.

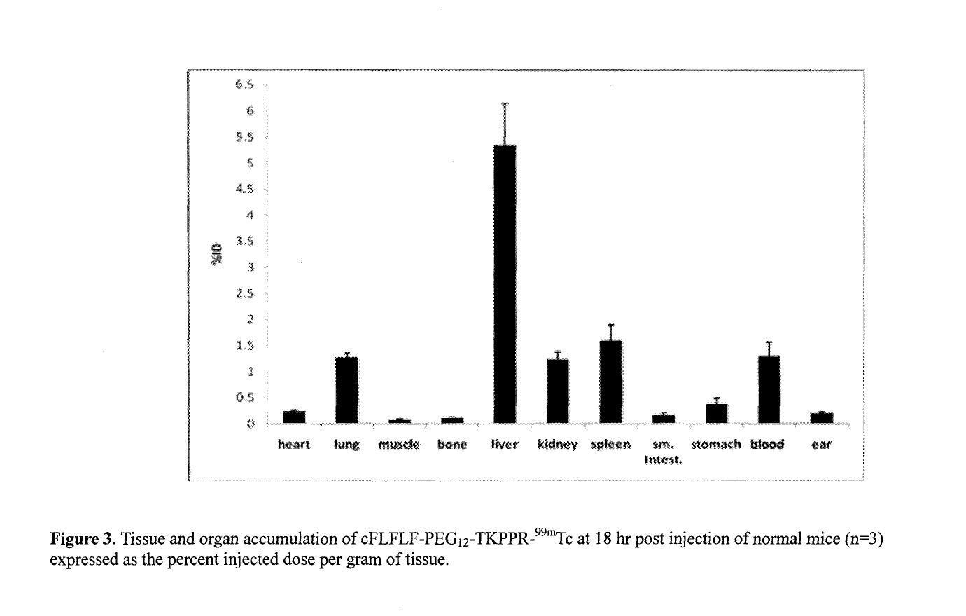

As shown in FIG. 3, biodistribution of cFLFLF-PEG₁₂-TKPPR-^{99m}Tc indeed showed higher liver uptake (5.3% ID) at 18 hours post injection in comparison to 1.5% ID of cFLFLFK-PEG-⁶⁴Cu(5). The longer blood retaining of cFLFLF-PEG₁₂-TKPPR-^{99m}Tc compared to cFLFLFK-PEG-⁶⁴Cu, 1.2% ID vs. 0.2% ID correlates the longer circulation time (T_{1/2}) of 84 vs. 55 min (FIG. 2).

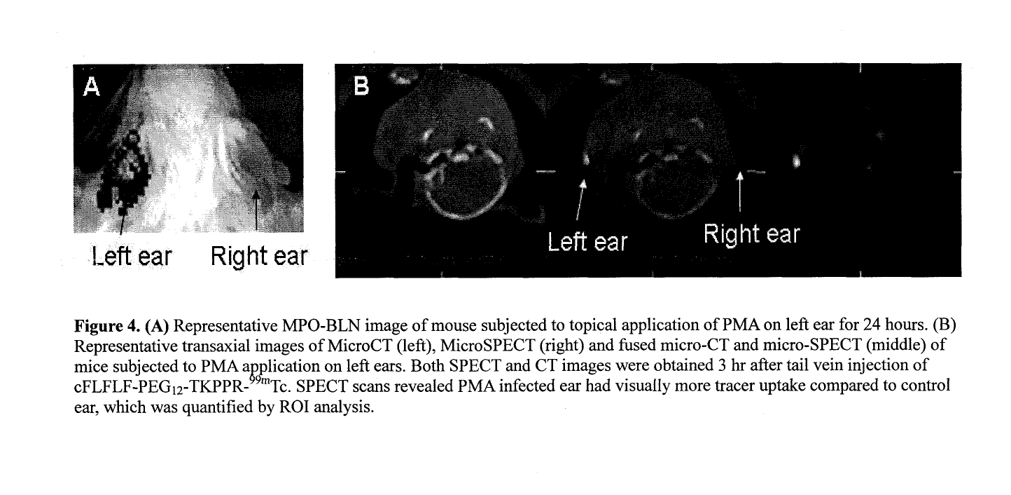

At 22 hours post PMA treatment, bioluminescence as observed by MPO assay was locally emitted from PMA-treated earlobes of FVB (female) mice after luminal administration, reaching levels of 7 fold over vehicle-treated ears. The image (FIG. 4A) of 21 min after luminal injection was chosen as a representative due to the highest ratio of photons emitted from infected ear (left) to control ear (right). At 27 hours after PMA challenge and 3 hours post injection of cFLFLF-PEG₁₂-TKPPR-^{99m}Tc, CT/SPECT imaging shows that the accumulation of the tracer measured in the infected ear (left) is about 3.15 folds higher than in the control ear (FIG. 4B).

These data suggest that in vivo detection of inflammation through neutrophil activation and migration in live animal is feasible with gamma or SPECT modalities.

### CONCLUSION

In conclusion, we have synthesized two neutrophil-targeting heterobivalent SPECT imaging probes: cFLFLF-PEG₄-TKPPR-^{99m}Tc and cFLFLF-PEG₁₂-TKPPR-^{99m}Tc. The peptide ligands bound to neutrophils with high affinity and demonstrated stability in serum and sufficient hydrophilicity. The cFLFLF-PEG₁₂-TKPPR-^{99m}Tc is a feasible probe for in vivo imaging of acute neutrophilic inflammation. On the basis of the in vivo imaging results and in vitro cell function assays, these peptides possess properties of promising new radiopharmaceuticals for in vivo imaging of neutrophils.

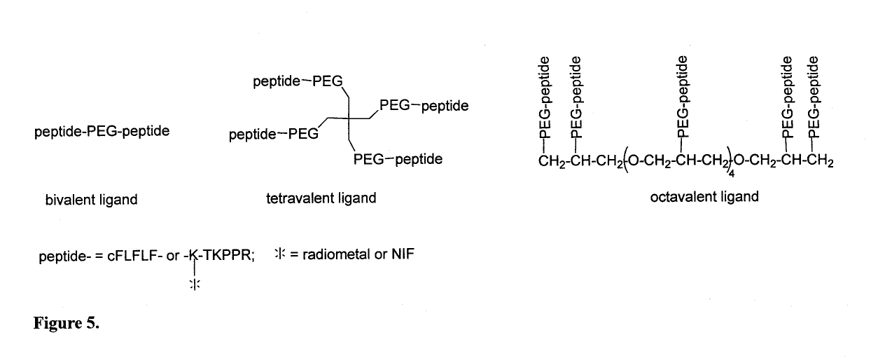

While the invention has been illustrate using two specific example probes, one skilled in the art would appreciate that modifications of these examples are possible without departing from the scope of the invention, for example, the particular lengths of the PEG linker can be modified or the PEG link may be linear or branched, the peptide sequences may be modified (e.g., homologous amino acid substitutions, deletions, or additions of amino acids as long as the binding ability is retained), or linking of these probes to form multi-valence probes as shown in FIG. 5.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

### BIBLIOGRAPHY

(1) PALESTRO, C. J. (2007) IN VIVO LEUKOCYTE LABELING: THE QUEST CONTINUES. J NUCL MED 48, 332-334.
(2) BABICH, J. W., TOMPKINS, R. G., GRAHAM, W., BARROW, S. A., AND FISCHMAN, A. J. (1997) LOCALIZATION OF RADIOLABELED CHEMOTACTIC PEPTIDE AT FOCAL SITES OF ESCHERICHIA COLI INFECTION IN RABBITS: EVIDENCE FOR A RECEPTOR-SPECIFIC MECHANISM. [SEE COMMENT]. JOURNAL OF NUCLEAR MEDICINE 38, 1316-22.
(3) BABICH, J. W., DONG, Q., GRAHAM, W., BARZANA, M., FERRIL, K., PIKE, M., AND FISCHMAN, A. J. (1997) A NOVEL HIGH AFFINITY CHEMOTACTIC PEPTIDE ANTAGONIST FOR INFECTION IMAGING. J NUCL MED 38, 268P.
(4) ZHANG, Y., KUNDU, B., FAIRCHILD, K. D., LOCKE, L., BERR, S. S., LINDEN, J., AND PAN, D. (2007) SYNTHESIS OF NOVEL NEUTROPHIL-SPECIFIC IMAGING AGENTS FOR POSITRON EMISSION TOMOGRAPHY (PET) IMAGING. BIOORGANIC & MEDICINAL CHEMISTRY LETTERS 17, 6876-6878.
(5) LOCKE, L. W., CHORDIA, M. D., ZHANG, Y., KUNDU, B., KENNEDY, D., LANDSEADEL, J., XIAO, L., FAIRCHILD, K. D., BERR, S. S., LINDEN, J., AND PAN, D. (2009) A NOVEL NEUTROPHIL-SPECIFIC PET IMAGING AGENT: CFLFLFK-PEG-64CU. J NUCL MED 50, 790-797.
(6) XIAO, L., ZHANG, Y., LIU, Z., YANG, M., PU, L., AND PAN, D. SYNTHESIS OF THE CYANINE 7 LABELED NEUTROPHIL-SPECIFIC AGENTS FOR NONINVASIVE NEAR INFRARED FLUORESCENCE IMAGING. BIOORGANIC & MEDICINAL CHEMISTRY LETTERS 20, 3515-3517.
(7) RAO, J., LAHIRI, J., ISAACS, L., WEIS, R. M., AND WHITESIDES, G. M. (1998) A TRIVALENT SYSTEM FROM VANCOMYCIN-D-ALA-D-ALA WITH HIGHER AFFINITY THAN AVIDIN-BIOTIN. SCIENCE 280, 708-711.
(8) FAN, E., ZHANG, Z., MINKE, W. E., HOU, Z., VERLINDE, C. L. M. J., AND HOL, W. G. J. (2000) HIGH-AFFINITY PENTAVALENT LIGANDS OF <I>ESCHERICHIA COLI</I> HEAT-LABILE ENTEROTOXIN BY MODULAR STRUCTURE-BASED DESIGN. J. AM. CHEM. SOC. 122, 2663-2664.
(9) MAMMEN, M., CHOI, S. K., AND WHITESIDES, G. M. (1998) POLYVALENT INTERACTIONS IN BIOLOGICAL SYSTEMS: IMPLICATIONS FOR DESIGN AND USE OF MULTIVALENT LIGANDS AND INHIBITORS. ANGEWANDTE CHEMIE-INTERNATIONAL EDITION 37, 2755-2794.
(10) KIESSLING, L. L., GESTWICKI, J. E., AND STRONG, L. E. (2000) SYNTHETIC MULTIVALENT LIGANDS IN THE EXPLORATION OF CELL-SURFACE INTERACTIONS. CURRENT OPINION IN CHEMICAL BIOLOGY 4, 696-703.
(11) KIESSLING, L. L., STRONG, L. E., AND GESTWICKI, J. E. (2000) PRINCIPLES FOR MULTIVALENT LIGAND DESIGN, IN ANNUAL REPORTS IN MEDICINAL CHEMISTRY, VOL 35 PP 321-330.
(12) KITOV, P. I., SADOWSKA, J. M., MULVEY, G., ARMSTRONG, G. D., LING, H., PANNU, N. S., READ, R. J., AND BUNDLE, D. R. (2000) SHIGA-LIKE TOXINS ARE NEUTRALIZED BY TAILORED MULTIVALENT CARBOHYDRATE LIGANDS. NATURE 403, 669-672.
(13) ZHANG, Y., XIAO, L., CHORDIA, M. D., LOCKE, L. W., WILLIAMS, M. B., BERR, S. S., AND PAN, D. NEUTROPHIL TARGETING HETEROBIVALENT SPECT IMAGING PROBE: CFLFLF-PEG-TKPPR-99MTC. BIOCONJUGATE CHEMISTRY 21, 1788-1793.
(14) TWEEDLE, M. F. (2006) ADVENTURES IN MULTIVALENCY, THE HARRY S. FISCHER MEMORIAL LECTURE CMR 2005; EVIAN, FRANCE. CONTRAST MEDIA & MOLECULAR IMAGING 1, 2-9.
(15) FERRANTE, A., AND THONG, Y. H. (1980) SIMULTANEOUS PREPARATION OF MONONUCLEAR AND POLYMORPHONUCLEAR LEUCOCYTES FROM HORSE BLOOD ON FICOLL-HYPAQUE MEDIUM. JOURNAL OF IMMUNOLOGICAL METHODS 34, 279-285.
(16) SULLIVAN, G. W., RIEGER, J. M., SCHELD, W. M., MACDONALD, T. L., AND LINDEN, J. (2001) CYCLIC AMP-DEPENDENT INHIBITION OF HUMAN NEUTROPHIL OXIDATIVE ACTIVITY BY SUBSTITUTED 2-PROPYNYLCYCLOHEXYL ADENOSINE A(2A) RECEPTOR AGONISTS. BRITISH JOURNAL OF PHARMACOLOGY 132, 1017-26.
(17) STOLIN, A., WOJCIK, R., AND WILLIAMS, M. (2006) IN CONFERENCE RECORD OF THE IEEE NUCLEAR SCIENCE SYMPOSIUM AND MEDICAL IMAGING CONFERENCE, SAN DIEGO CA.
(18) ZHENG, Y., LI, H., WANG, J., STOLIN, A. V., POLE, D. J., AND WILLIAMS, M. B. (2007) IN PROCEEDINGS OF SPIE PP 649804-1-649804-11, SAN JOSE, CA.
(19) GROSS, S., GAMMON, S. T., MOSS, B. L., RAUCH, D., HARDING, J., HEINECKE, J. W., RATNER, L., PIWNICA-WORMS, D., GROSS, S., GAMMON, S. T., MOSS, B. L., RAUCH, D., HARDING, J., HEINECKE, J. W., RATNER, L., AND PIWNICA-WORMS, D. (2009) BIOLUMINESCENCE IMAGING OF MYELOPEROXIDASE ACTIVITY IN VIVO. NATURE MEDICINE 15, 455-61.
(20) LIU, J., WANG, Z. T., JI, L. L., LIU, J., WANG, Z.-T., AND JI, L.-L. (2007) IN VIVO AND IN VITRO ANTI-INFLAMMATORY ACTIVITIES OF NEOANDROGRAPHOLIDE. AMERICAN JOURNAL OF CHINESE MEDICINE 35, 317-28.
(21) FRETLAND, D., GOKHALE, R., MATHUR, L., BARON, D., PAULSON, S., AND STOLZENBACH, J. (1995) DERMAL INFLAMMATION IN PRIMATES, MICE, AND GUINEA PIGS ATTENUATION BY SECOND-GENERATION LEUKOTRIENE B4 RECEPTOR ANTAGONIST, SC-53228. INFLAMMATION 19, 333-46.

1. A nuclear or NI (near infrared) optical imaging agent for imaging a site of inflammation in a mammalian body, the imaging agent comprising two neutrophil-binding peptide sequences linked by a hydrophilic polyethyleneglycol (PEG) moiety, and a radionuclide or a near infrared fluorophore (NIF).

2. The imaging agent of claim 1, wherein the neutrophile-binding peptide sequences comprise a formyl peptide receptor (FPR) binding sequence, cF(D)LF(D)LF, or a tuftsin receptor binding sequence, TKPPR.

3. The imaging agent of claim 1, wherein the PEG is either linear or branched with a molecular weight from 500 to 10,000.

4. The imaging agent of claim 1, wherein the radionuclide is conjugated with the imaging reagent via a radiometal chelator or is directly coupled with a covalent bond.

5. The imaging agent of claim 1, wherein the NIF is coupled with the imaging reagent via a covalent amide bond.

6. The imaging agent of claim 1, wherein the radionuclide is one of the following: ^{99m}Tc, ¹¹¹In, ¹²⁵I, ¹²³I, ¹³¹I, ¹²⁴I, ⁶⁷Ga, ⁶⁸Ga, or ⁶⁴Cu.

7. The imaging agent of claim 1, wherein the NIF is one of the following: cyanine 5.5, cyanine 7, cyanine 7.5, or indocyanine green.

8. The imaging agent of claim 3, wherein the linear or branched PEG moiety is couple with neutrophil-binding peptide sequences, cF(D)LF(D)LF or TKPPR, on a terminal end of each branch of the PEG.

9. The imaging agent of claim 2, wherein the PEG is either linear or branched with a molecular weight from 500 to 10,000.

10. The imaging agent of claim 2, wherein the radionuclide is conjugated with the imaging reagent via a radiometal chelator or is directly coupled with a covalent bond.

11. The imaging agent of claim 3, wherein the radionuclide is conjugated with the imaging reagent via a radiometal chelator or is directly coupled with a covalent bond.

12. The imaging agent of claim 2, wherein the NIF is coupled with the imaging reagent via a covalent amide bond.

13. The imaging agent of claim 3, wherein the NIF is coupled with the imaging reagent via a covalent amide bond.

14. The imaging agent of claim 2, wherein the radionuclide is one of the following: ^{99m}Tc, ¹¹¹In, ¹²⁵I, ¹²³I, ¹³¹I, ¹²⁴I, ⁶⁷Ga, ⁶⁸Ga, or ⁶⁴Cu.

15. The imaging agent of claim 3, wherein the radionuclide is one of the following: ^{99m}Tc, ¹¹¹In, ¹²⁵I, ¹²³I, ¹³¹I, ¹²⁴I, ⁶⁷Ga, ⁶⁸Ga, or 64Cu.

16. The imaging agent of claim 4, wherein the radionuclide is one of the following: ^{99m}Tc, ¹¹¹In, ¹²⁵I, ¹²³I, ¹³¹I, ¹²⁴I, ⁶⁷Ga, ⁶⁸Ga, or ⁶⁴Cu.

17. The imaging agent of claim 2, wherein the NIF is one of the following: cyanine 5.5, cyanine 7, cyanine 7.5, or indocyanine green.

18. The imaging agent of claim 3, wherein the NIF is one of the following: cyanine 5.5, cyanine 7, cyanine 7.5, or indocyanine green.

19. The imaging agent of claim 5, wherein the NIF is one of the following: cyanine 5.5, cyanine 7, cyanine 7.5, or indocyanine green.
